(19) United States
(12) Patent Application Publication
(10) Pub. No.: US 2026/0183223 A1
(43) Pub. Date: Jul. 2, 2026

(54) A PERSONAL CARE COMPOSITION

(71) Applicant: Conopco, Inc., d/b/a UNILEVER, Hoboken, NJ (US)

(72) Inventors: Xuelan GU, Shanghai (CN); Xue XIAO, Shanghai (CN)

(73) Assignee: Conopco, Inc., d/b/a UNILEVER, Hoboken, NJ (US)

(21) Appl. No.: 19/128,054

(22) PCT Filed: Oct. 17, 2023
(86) PCT No.: PCT/EP2023/078719

§ 371 Date: May 7, 2025

(30) Foreign Application Priority Data
Nov. 11, 2022 (WO) PCT/CN2022/131262
Dec. 9, 2022 (EP) 22212382.0

(51) Int. Cl. A61K 8/9789; A61K 8/44; A61K 8/49; A61K 8/9728; A61Q 19/08

(52) U.S. Cl. CPC A61K 8/9789; A61K 8/447; A61K 8/4973; A61K 8/9728; A61Q 19/08

(57) ABSTRACT

Disclosed is a personal care composition comprising carboxymethyl cysteine compound and a source of Atractylenolide.

### FIELD OF THE INVENTION

The present invention relates to a personal care composition comprising carboxymethyl cysteine compound and a source of Atractylenolide. It was surprisingly found that the expression of collagen type III alpha I chain was significantly enhanced by combining carboxymethyl cysteine compound and a source of Atractylenolide.

### BACKGROUND OF THE INVENTION

The skin is a primary barrier of the human body. It protects the organs in the body from external stimulations. It is subject to intrinsic aging and extrinsic aging. Skin aging is a complex process, and alterations in human skin due to aging have distinct characteristics as compared to other organs. Characteristics of intrinsic or chronological skin aging include dryness, visible free lines and wrinkles, uneven skin pigmentation, loss of elasticity and skin sagging. Extrinsic factors including exposure to sunlight, pollutants and cigarette smoke can accelerate the skin aging process, especially on the face.

Collagen, the predominant matrix of the skin protein, is known to impart tensile strength to skin. It has been shown that collagen is significantly reduced with age and UV exposure. The degradation or destruction of the architecture of collagen decreases the tensile strength of the skin and therefore causing wrinkles and laxity. Collagen destruction is thought to underlie the characteristic alterations in the appearance of aged skin.

One of the most efficient ways for providing the effect of anti-aging is to promote the production of collagen. Upregulating the expression of collagen synthesizing genes typically leads to an increase in collagen production.

Therefore, the present inventors have recognized that there is a need to develop solution to enhance the expression of collagen synthesizing genes, for example collagen type III alpha I chain (COL3A1). It was surprisingly found that by combining carboxymethyl cysteine and a source of Atractylenolide, the expression of COL3A1 was synergistically upregulated.

### SUMMARY OF THE INVENTION

In a first aspect, the present invention is directed to a personal care composition comprising carboxymethyl cysteine compound and a source of Atractylenolide.

In a second aspect, the present invention is directed to a method of providing the skin benefits selected from the group consisting of enhancing collagen production in the skin, improving skin elasticity, reducing the appearance of wrinkles reducing sagging, and anti-aging comprising a step of topically applying to the skin the composition of the composition of the present invention.

In a third aspect, the present invention is directed to use of the composition of the present invention for providing the skin benefits selected from the group consisting of enhancing collagen production in the skin, improving skin elasticity, reducing the appearance of wrinkles reducing sagging, and anti-aging.

All other aspects of the present invention will more readily become apparent upon considering the detailed description and examples which follow.

### DETAILED DESCRIPTION OF THE INVENTION

Except in the examples, or where otherwise explicitly indicated, all numbers in this description indicating amounts of material or conditions of reaction, physical properties of materials and/or use may optionally be understood as modified by the word “about”.

All amounts are by weight of the composition, unless otherwise specified.

It should be noted that in specifying any range of values, any particular upper value can be associated with any particular lower value.

For the avoidance of doubt, the word “comprising” is intended to mean “including” but not necessarily “consisting of” or “composed of”. In other words, the listed steps or options need not be exhaustive.

The disclosure of the invention as found herein is to be considered to cover all embodiments as found in the claims as being multiply dependent upon each other irrespective of the fact that claims may be found without multiple dependency or redundancy.

Where a feature is disclosed with respect to a particular aspect of the invention (for example a composition of the invention), such disclosure is also to be considered to apply to any other aspect of the invention (for example a method of the invention) mutatis mutandis.

The carboxymethyl cysteine compound refers to compound selected from carboxymethyl cysteine, salt of carboxymethyl cysteine, ester of carboxymethyl cysteine, amide of carboxymethyl cysteine or a mixture thereof. Preferably, the carboxymethyl cysteine compound comprises carboxymethyl cysteine, ester of carboxymethyl cysteine, and/or salt of carboxymethyl cysteine. More preferably, the carboxymethyl cysteine compound comprises carboxymethyl cysteine, and/or salt of carboxymethyl cysteine. Even more preferably, carboxymethyl cysteine compound comprises salt of carboxymethyl cysteine. Still even more preferably the carboxymethyl cysteine compound comprises lysine carboxymethyl cysteinate and most preferably, the carboxymethyl cysteine compound is lysine carboxymethyl cysteinate.

Preferably, the carboxymethyl cysteine compound is present in amount of at least 0.00001%, more preferably at least 0.0001%, even more preferably at least 0.001%, still even more preferably at least 0.01%, and most preferably at least 0.1% by weight of the composition. Preferably, the carboxymethyl cysteine compound is present in amount of no greater than 10%, more preferably no greater than 5%, even more preferably no greater than 3%, still even more preferably no greater than 1%, and most preferably no greater than 0.5% by weight of the composition.

Preferably, the lysine carboxymethyl cysteinate is present in amount of no greater than 10%, more preferably no greater than 5%, even more preferably no greater than 3%, still even more preferably no greater than 1%, and most preferably no greater than 0.5% by weight of the composition. Preferably, the lysine carboxymethyl cysteinate is present in amount of at least 0.00001%, more preferably at least 0.0001%, even more preferably at least 0.001%, still even more preferably at least 0.01%, and most preferably at least 0.1% by weight of the composition.

The term “source of Atractylenolide” as used herein refers to a substance, blend or mixture comprising the active ingredient Atractylenolide. In case of present invention, the sources of Atractylenolide preferably refer to Atractylenolide per se or a plant extract comprising Atractylenolide. Plant extract as used herein refers to extract derived from a plant or parts of a plant such as roots, stem, leaf, fruit, bark and/or flower. Where the plant is a fungus, such as mushroom, then the material is preferably derived from the sclerotium. Preferably, the source of Atractylenolide is source of Atractylenolide-I.

Atractylenolide is a botanical active, found in the rhizome of a plant of Atractylodes origin. The term “botanical active” preferably refers to a compound present in a plant which provides a particular benefit e.g. pain relief, anti-inflammation benefit, work as antioxidant etc. In practice, botanical actives are extracted from the plant or in some cases plant extract itself is used in a composition for providing benefits.

In a preferred embodiment, the source of Atractylenolide is the Atractylenolide per se. Preferably, the source of Atractylenolide is Atractylenolide-I per se. Preferably. The amount of Atractylenolide is preferably in the range of 0.00000001 to 3% by weight, more preferably 0.0000001 to 0.1% by weight, even more preferably 0.000001 to 0.02% by weight and most preferably 0.00001 to 0.005% by weight of the composition. Preferably. The amount of Atractylenolide-I is preferably in the range of 0.00000001 to 3% by weight, more preferably 0.0000001 to 0.1% by weight, even more preferably 0.000001 to 0.02% by weight and most preferably 0.00001 to 0.005% by weight of the composition.

In another preferred embodiment, the source of Atractylenolide is a plant extract comprising Atractylenolide, preferably Atractylenolide-I. Preferably, the source of Atractylenolide is an aqueous plant extract comprising Atractylenolide, preferably Atractylenolide-I. The term “aqueous extract” means that the extract is obtained by using water, or water mixing with other solvent as the solvent for extraction. However, it is preferable that the aqueous extract is obtained by solely using water as solvent for extraction. Other solvent may be selected from ethanol, acetone, ethyl acetate, glycerin, butylene glycol or a mixture thereof. Preferably, the aqueous extract is solid at 25° C. and atmospheric pressure.

More preferably the source of Atractylenolide is plant extract of Atractylodes macrocephala, preferably aqueous plant extract of Atractylodes macrocephala. Even more preferably, the source of Atractylenolide is plant extract of the root of Atractylodes macrocephala. Still even more preferably, the source of Atractylenolide is plant extract, preferably aqueous plant extract, of combination of Atractylodes macrocephala, Paeonia lactiflora, Poria cocos and Glycyrrhiza glabra. For sake of clarity, the plant extract of a combination of Atractylodes macrocephala, Paeonia lactiflora, Poria cocos and Glycyrrhiza glabra may be obtained by extracting the combination together, and/or extracting Atractylodes macrocephala, Paeonia lactiflora, Poria cocos, Glycyrrhiza glabra or any mixture thereof and combining them together.

Preferably, the amount of the plant extract is preferably in the range of 0.00001 to 15% by weight, more preferably 0.0001 to 9% by weight, even more preferably 0.005 to 4% by weight and most preferably 0.1 to 2% by weight of the composition. Preferably, the amount of the plant extract of Atractylodes macrocephala is preferably in the range of 0.00001 to 15% by weight, more preferably 0.0001 to 9% by weight, even more preferably 0.005 to 4% by weight and most preferably 0.1 to 2% by weight of the composition. Preferably, the amount of the plant extract of Atractylodes macrocephala is preferably in the range of 0.00001 to 15% by weight, more preferably 0.0001 to 9% by weight, even more preferably 0.005 to 4% by weight and most preferably 0.1 to 2% by weight of the composition. Preferably, the amount of the plant extract of combination of Atractylodes macrocephala, Paeonia lactiflora, Poria cocos and Glycyrrhiza glabra is preferably in the range of 0.00001 to 15% by weight, more preferably 0.0001 to 9% by weight, even more preferably 0.005 to 4% by weight and most preferably 0.1 to 2% by weight of the composition. For sake of clarity, the weight of the plant extract in the present invention typically refers to the weight of solid extracted from the plant.

When a plant extract comprising Atractylenolide is used in the composition, the concentration of the plant extract may be adjusted to aforesaid range depending on active level of Atractylenolide in the plant extract.

Preferably, the weight ratio of the carboxymethyl cysteine compound to the Atractylenolide is 1:500 to 50:1, more preferably 1:150 to 20:1, even more preferably 1:60 to 8:1, still even more preferably 1:25 to 3:1 and most preferably 1:8 to 1:1. Preferably, the weight ratio of the lysine carboxymethyl cysteinate to the Atractylenolide is 1:500 to 50:1, more preferably 1:150 to 20:1, even more preferably 1:60 to 8:1, still even more preferably 1:25 to 3:1 and most preferably 1:8 to 1:1.

Preferably, the weight ratio of the carboxymethyl cysteine compound to the plant extract of Atractylodes macrocephala is 1:20000 to 1:1, more preferably 1:8000 to 1:2 and even more preferably 1:2000 to 1:10. Preferably, the weight ratio of the lysine carboxymethyl cysteinate to the plant extract of Atractylodes macrocephala is 1:20000 to 1:1, more preferably 1:8000 to 1:2 and even more preferably 1:2000 to 1:10.

Preferably, the weight ratio of the carboxymethyl cysteine compound to the plant extract of combination of Atractylodes macrocephala, Paeonia lactiflora, Poria cocos and Glycyrrhiza glabra is 1:50000 to 1:2, more preferably 1:20000 to 1:10, even more preferably 1:5000 to 1:50 and most preferably 1:2000 to 1:100. Preferably, the weight ratio of the lysine carboxymethyl cysteinate to the plant extract of combination of Atractylodes macrocephala, Paeonia lactiflora, Poria cocos and Glycyrrhiza glabra is 1:50000 to 1:2, more preferably 1:20000 to 1:10, even more preferably 1:5000 to 1:50 and most preferably 1:2000 to 1:100.

The composition may optionally comprise whitening pigment. Whitening pigments are typically particles of high refractive index materials. For example, the whitening pigment may have a refractive index of greater than 1.3, more preferably greater than 1.8 and most preferably from 2.0 to 2.7. Examples of such whitening pigment are those comprising bismuth oxy-chloride, boron nitride, barium sulfate, mica, silica, titanium dioxide, zirconium oxide, aluminium oxide, zinc oxide or combinations thereof. More preferred whitening pigment are particles comprising titanium dioxide, zinc oxide, zirconium oxide, mica, iron oxide or a combination thereof. Even more preferred whitening pigment are particles comprising zinc oxide, zirconium oxide, titanium dioxide or a combination thereof as these materials have especially high refractive index. Still even more preferably the whitening pigment is selected from titanium dioxide, zinc oxide or a mixture thereof and most preferred whitening pigment is titanium dioxide. The average diameter of whitening pigment is typical from 15 nm to 1 micron, more preferably from 35 nm to 800 nm, even more preferably from 50 nm to 500 nm and still even more preferably from 100 to 300 nm. Amount of whitening pigment may be 0.1 to 15%, preferably 0.5 to 5% by weight of the composition.

Preferably, the composition comprises a glutamate source selected from the group consisting of glutamine, glutamine ester, glutamic acid, pyroglutamic acid, salts, and mixtures thereof. More preferably, the composition comprises pyroglutamic acid and/or salt of pyroglutamic acid. Even more preferably, the composition comprises sodium salt of pyroglutamic acid. Preferably, the glutamate source is present in amount of 0.0001 to 10% by weight of the composition, more preferably 0.001 to 6%, even more preferably 0.01 to 3% by weight of the composition.

Preferably, the composition comprises polyhydric alcohol. Polyhydric alcohols may be selected from group of glycerin, propylyene glycol, dipropylene glycol, polypropylene glycol, polyethylene glycol, sorbitol, hydroxypropyl sorbitol, hexylene glycol, 1,3-butylene glycol, isoprene glycol, ethoxylated glycerol, propoxylated glycerol or a mixture thereof. Most preferred polyhydric alcohol is glycerol known also as glycerin. The amount of polyhydric alcohol may range anywhere from 0.1 to 20%, preferably 0.5 to 15% and more preferably 2 and 10% by weight of the composition.

Preferably, the composition comprises emollient materials. Suitable emollient materials include silicones, hydrocarbons, triglycerides or a mixture thereof. These silicones may be organic, silicone-containing or fluorine-containing, volatile or non-volatile, polar or non-polar. Hydrocarbons may include mineral oil, petrolatum and polyalpha-olefins. Examples of preferred volatile hydrocarbons include polydecanes such as isododecane and isodecane (e.g. Permethyl-99A which is available from Presperse Inc.) and the C7-C8 through C12-C15 isoparaffins (such as the Isopar Series available from Exxon Chemicals). Illustrative triglycerides but not limiting are sunflower seed oil, cotton oil, canola oil, soybean oil, castor oil, borage oil, olive oil, shea butter, jojoba oil and mixtures thereof. Mono- and di-glycerides may also be useful. Particularly preferable are glyceryl monostearate and glyceryl distearate.

Preferably, the composition comprises moisturizing agents. Particularly preferred moisturizing agents includes, petrolatum, aquaporin manipulating actives, oat kernel flour, substituted urea like hydroxyethyl urea, hyaluronic acid and/or its precursor N-acetyl glucosamine, hyaluronic acid and/or its precursor N-acetyl glucosamine, or a mixture thereof.

Some compositions may include thickeners. These may be selected from cellulosics, natural gums and acrylic polymers but not limited by this thickening agent types. Among the cellulosics are sodium carboxymethyl cellulose, hydroxyethyl cellulose, hydroxypropyl methylcellulose and combinations thereof. Suitable gums include xanthan, pectin, karaya, agar, alginate gums and combinations thereof. Among the acrylic thickeners are homopolymers and copolymers of acrylic and methacrylic acids including carbomers such as Carbopol 1382, Carbopol 982, Ultrez, Aqua SF-1 and Aqua SF-2 available from the Lubrizol Corporation. Amounts of thickener may range from 0.01 to 3% by weight of the active polymer (outside of solvent or water) in the compositions.

In addition, the compositions of the invention may further include 0.5 to 10% by weight of sequestering agents, such as tetra sodium ethylenediaminetetraacetate (EDTA), EHDP or mixtures; opacifiers and pearlizers such as ethylene glycol distearate, titanium dioxide or Lytron 621 (Styrene/Acrylate copolymer); all of which are useful in enhancing the appearance or properties of the product.

The composition may comprise water in amount of 10 to 96% by weight of the composition, more preferably from 25 to 92%, even more preferably from 42 to 88%, most preferably from 55 to 82% by weight of the composition.

Preferably, the composition has a viscosity of at least 10 mPa·s, more preferably in the range 30 to 10000 mPa·s, even more preferably 50 to 5000 mPa·s, and most preferably 100 to 2000 mPa·s, when measured at 20 degrees C. at a relatively high shear rate of about 20 s⁻¹.

Preferably, the composition is an emulsion, more preferably an oil-in-water emulsion. Preferably the composition is a fluid liquid at 25° C. and atmospheric pressure.

Preferably, the personal care composition is a skin care composition. Skin care composition refers to a composition suitable for topical application to human skin, including leave-on and wash-off products but preferably leave-on compositions. The term “leave-on” as used with reference to compositions herein means a composition that is applied to or rubbed on the skin, and left thereon. The term “wash-off” as used with reference to compositions herein means a skin cleanser that is applied to or rubbed on the skin and rinsed off substantially immediately subsequent to application. The term “skin” as used herein includes the skin on the face, neck, chest, abdomen, back, arms, under arms, hands, and legs. Preferably “skin” means includes the skin on the face and under arms, more preferably skin means skin on the face other than lips and eyelids. The composition is particularly preferably a moisturizer rather than a make-up product.

Preferably, the composition is a topical composition. Preferably, the composition may be in the form of cream, lotion, ointment, solution, suspension, emulsion, paste, gel, powder, powder foundation, emulsion foundation, wax foundation, or spray. More preferably, the composition may be formulated in the form of cream, lotion, ointment, emulsion, gel, or a spray.

Preferably the use is non-therapeutic. Preferably the method is non-therapeutic. The term non-therapeutic typically means for cosmetic purposes and not curative or therapeutic purposes.

Preferably, the composition is capable of upregulating the expression of collagen type III alpha I chain (COL3A1) gene by at least 1.3 fold change, more preferably 1.3 to 5 and most preferably 1.4 to 3.5 and most preferably 1.6 to 2.5 fold change, typically in comparison to personal care composition comprising neither carboxymethyl cysteine compound source of Atractylenolide.

The following examples are provided to facilitate an understanding of the invention. The examples are not intended to limit the scope of the claims.

### EXAMPLES

### Materials

| Supplier | Trade name | Active | Abbreviation | Active wt % |
|---|---|---|---|---|
| Sinerga | HAIR APP | Lysine | LCC | >98% |
| | | carboxymethyl | | |
| | | cysteinate | | |

### Example 1

This Example demonstrates the synergistic upregulation of gene expression by combining lysine carboxymethyl cysteinate and plant extract containing Atractylenolide.

### 1. Preparation of San-Bai-Tang Extract (SBT)

An aqueous co-extract of the Atractylodes macrocephala, Paeonia lactiflora, Poria cocos and Glycyrrhiza glabra in the composition ratio of 2:2:2:1 parts by weight was prepared by the usual water-reflux process in which the duration of each reflux cycle was 30 minutes. There were two such cycles. A rotary evaporator maintained at 60° C. was used for vacuum distillation. The aqueous extract was freeze-dried into a powder. The extraction yield was 20:1 part by weight. The freeze-dried extract contains 505 ppm of total Atractylenolide.

### 2. Gene Expression Test

The normal human dermal fibroblasts (NHDF, Biocell, Xi′an, China, Cat: Fb20081902) were incubated in medium together with or without actives for 48 hours. After incubation, the total RNA for each NHEK was extracted using RNAex Pro reagent (Accurate Biotechnology, Cat: AG21102) according to manufacturer's protocol.

The extracted RNA was quantified using Nanodrop 2000 spectrometer (Thermo Fisher Scientific, Waltham, MA, US) and reverse transcribed to generate the template cDNA using the Evo M-MLV RT Premix for qPCR (Accurate Biotechnology, Cat: AG11706) according to manufacturer's protocol. Amplification of the gene encoding collagen type III alpha I chain (COL3A1) gene was performed with RT-PCR reagent (SYBR® Green Premix Pro Taq HS qPCR Kit, Accurate Biotechnology, Cat: AG11701) on the ABI Vii 7 Real-Time PCR Systems (Applied Biosystems, Thermo fisher scientific, Carlsbad, CA, USA). The beta-actin (ACTB) gene was selected as the housekeeping gene and all data on relative expression of the target genes was normalized to ACTB gene. The fold changes in expression were calculated relative to the blank control (medium having no active). All tests were conducted at least three times and Table 1 shows the fold changes in gene expression of COL3A1.

TABLE 1

| | | Fold change of expression |
|---|---|---|
| | Active* | of COL3A1 |
| Control | — | 1.0 |
| A | 0.000325 wt % of LCC | 1.1 |
| B | 0.5 wt % of SBT | 1.0 |
| 1 | 0.000325 wt % of LCC + | 1.8 ^{a} |
| | 0.5 wt % of SBT | |

The level of actives is weight percentage based on the amount of medium
^{a} significantly better (p < 0.05) than either of single active with at same level.

As demonstrated in Table 1, it was surprisingly found that the fold change when combining lysine carboxymethyl cysteinate and SBT is significantly higher than the product of the fold change for lysine carboxymethyl cysteinate only and that for SBT only. The expression of COL3A1 gene synergistically upregulated by combining lysine carboxymethyl cysteinate and SBT.

1. A personal care composition comprising carboxymethyl cysteine compound and a source of Atractylenolide.

2. The composition according to claim 1, wherein the carboxymethyl cysteine compound comprises carboxymethyl cysteine, ester of carboxymethyl cysteine, and/or salt of carboxymethyl cysteine.

3. The composition according to claim 2, wherein the carboxymethyl cysteine compound comprises salt of carboxymethyl cysteine.

4. The composition according to claim 1, wherein the carboxymethyl cysteine compound is present in an amount of at least 0.00001% and no greater than 10% by weight of the composition.

5. The composition according to claim 1, wherein the source of Atractylenolide is Atractylenolide per se.

6. The composition according to claim 1, wherein the Atractylenolide is present in an amount in the range of 0.00000001 to 3% by weight of the composition.

7. The composition according to claim 1, wherein the carboxymethyl cysteine compound to the Atractylenolide is present in a weight ratio of 1:500 to 50:1.

8. The composition according to claim 1, wherein the source of Atractylenolide is a plant extract of Atractylodes macrocephala.

9. The composition according to claim 8, wherein the plant extract of Atractylodes macrocephala is present in an amount in the range of 0.00001 to 15% by weight of the composition.

10. The composition according to claim 8, wherein the carboxymethyl cysteine compound to the plant extract of Atractylodes macrocephala is present in a weight ratio of 1:20000 to 1:1.

11. The composition according to claim 8, wherein the carboxymethyl cysteine compound to the plant extract of Atractylodes macrocephala is present in a weight ratio of 1:2000 to 1:10.

12. The composition according to claim 1, wherein the composition is an emulsion.

13. The composition according to claim 1, wherein the composition comprises water in an amount of 10 to 96% by weight of the composition.

14. A method of providing the skin benefits selected from the group consisting of enhancing collagen production in the skin, improving skin elasticity, reducing the appearance of wrinkles reducing sagging, and anti-aging comprising a step of topically applying to the skin the composition of claim 1.

15. The composition according to claim 2, wherein the carboxymethyl cysteine compound comprises lysine carboxymethyl cysteinate.

16. The composition according to claim 1, wherein the carboxymethyl cysteine compound is present in an amount of at least 0.01% and no greater than 3% by weight of the composition.

17. The composition according to claim 1, wherein the amount of Atractylenolide is present in an amount in the range of 0.00001 to 0.005% by weight of the composition.

18. The composition according to claim 1, wherein the carboxymethyl cysteine compound to the Atractylenolide is present in a weight ratio of 1:25 to 3:1.

19. The composition according to claim 1, wherein the source of Atractylenolide is a plant extract of combination of Atractylodes macrocephala, Paeonia lactiflora, Poria cocos, and Glycyrrhiza glabra.

20. The composition according to claim 8, wherein the plant extract of Atractylodes macrocephala is present in an amount in the range of 0.005 to 4% by weight of the composition.
